(19) United States
(12) Patent Application Publication
(10) Pub. No.: US 2025/0387528 A1
(43) Pub. Date: Dec. 25, 2025

(54) Candle Warmer System and Method of Use

(71) Applicant: Christopher Barnhart, Davenport, IA (US)

(72) Inventor: Christopher Barnhart, Davenport, IA (US)

(21) Appl. No.: 19/169,047

(22) Filed: Apr. 3, 2025

Related U.S. Application Data
(60) Provisional application No. 63/661,676, filed on Jun. 19, 2024.

(51) Int. Cl. A61L 9/03

(52) U.S. Cl. CPC A61L 9/03; A61L 2209/11; A61L 2209/12; A61L 2209/21

(57) ABSTRACT

The present invention is a remote-operated candle warmer system. The system comprises a portable candle warming device which includes a heat-resistant housing, an electric heating plate for gently warming candles, and a power source that includes a rechargeable battery and an electric cord for dual power options. A plurality of flame-like LED illumination symbols on the housing visually indicates device activation. The system includes a remote-control device capable of wirelessly activating or deactivating the heating plate and illumination symbols within a predetermined range. The heating plate is temperature-regulated to ensure consistent warming, and the system incorporates safety features to prevent overheating. Additionally, the device can accommodate candles of varying wax types, sizes, and shapes.

### CROSS-REFERENCE TO RELATED APPLICATION

The present application claims priority to, and the benefit of, U.S. Provisional Application No. 63/661,676 which was filed on Jun. 19, 2024 and is incorporated herein by reference in its entirety.

### FIELD OF THE INVENTION

The present invention generally relates to candle warming systems. More specifically, the invention relates to a remote-operated candle warmer system. The system features a candle warming device with a heat-resistant housing and an electric heating plate that gently warms candles to release their fragrance without combustion. The system includes a wireless remote-control device for activating and deactivating the warming plate and LED illumination symbols from a distance, reducing the need for physical interaction with the device. The warming device is powered by a rechargeable battery or a standard electrical cord. Accordingly, the present disclosure makes specific reference thereto. Nonetheless, it is to be appreciated that aspects of the present invention are also equally applicable to other like applications, devices, and methods of manufacture.

### BACKGROUND

By way of background, candle warmers have become a popular alternative to traditional candles with open flames and offer a safer method to dispense candle scents in homes, offices, and other spaces. By using a heating plate to gently warm candles, these devices eliminate the risk of fire hazards associated with burning candles. Despite their advantages, standard candle warmers come with certain limitations and safety concerns.

One of the primary limitations of standard candle warmers is their reliance on manual activation, requiring users to position the device in easily accessible locations. This often leads to placement on countertops, shelves, or other open surfaces, where they may be vulnerable to accidental interference. In households with pets or children, this can pose significant safety risks. Curious pets or children might pull on the device's electrical cord, leading to dangerous spills of hot wax, which can cause burns, injuries, or property damage. Additionally, these devices often lack modern convenience features, such as remote operation or safety indicators, leaving users to monitor the warmer closely to avoid accidents. Furthermore, conventional candle warmers are typically limited to a single functionality, offering little adaptability or control over the heating process. Individuals desire an improved candle warmer device that overcomes the shortcomings of conventional candle warmer devices.

Therefore, there exists a long-felt need in the art for a candle warmer system that can be operated remotely to enhance safety and convenience for users. There is a long-felt need for a system that eliminates the risks associated with traditional open-flame candles while addressing the limitations of conventional candle warmers. Additionally, there is a long-felt need for a candle warming device that can be controlled remotely, reducing the potential for accidents caused by children or pets interacting with electrical cords or the device itself. Furthermore, there is a long-felt need for a system that provides safe use and energy efficiency. More specifically, there exists a long-felt need in the art for a remote-operated candle warmer system that offers wireless control, enabling users to safely and efficiently enjoy candle fragrances in a variety of environments. Finally, there is a long-felt need for a device that integrates modern technology, such as wireless remote operation and offers a much safer alternative to standard candle warmers.

The subject matter disclosed and claimed herein, in one embodiment, comprises a remote-operated candle warmer system that addresses the aforementioned needs. The system includes a candle warming device featuring a heat-resistant housing with an integrated electric heating plate configured to gently warm candles, releasing their fragrance without combustion. The warming device is powered by a rechargeable battery or a standard electrical cord, offering dual power options for flexibility. A signal receiver within the device processes wireless signals transmitted from a handheld remote-control device, enabling users to activate and deactivate the heating plate and illumination symbols from a safe distance. The illumination symbols, designed as flame-like LED indicators, provide a visual confirmation of the device's operational status. The system also incorporates advanced safety features, such as temperature regulation to prevent overheating and a durable, non-slip design to minimize accidental tipping.

In this manner, the remote-operated candle warmer system of the present invention accomplishes all of the foregoing objectives and provides a comprehensive solution for safe and convenient fragrance dispersion. The system reduces the risk of accidents caused by physical interaction with the device, especially in households with children or pets. The remote-control functionality enables users to activate the warmer from hard-to-reach locations, enhancing safety and user convenience. The use of flame-like LED indicators provides status of the device and the dual power options, and portable design make it suitable for use in diverse environments. The system of the invention significantly improves the usability, safety, and versatility of candle warming devices, providing an enhanced alternative to traditional methods of enjoying candle fragrances.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a remotely operated candle warmer system. The system comprises a candle warming device, the device includes a housing configured to store electronic components and constructed with heat-resistant materials, an electric heating plate disposed within the housing and configured to warm a candle placed thereon to release fragrance without combustion, an internal rechargeable battery for providing electric power to the electric heating plate, an electric cord including a plug for coupling the candle warming device to an external electrical supply, and a plurality of illumination symbols disposed on an exterior surface of the housing, the illumination symbols configured to indicate activation of the candle warming device, and a remote-control device configured to wirelessly transmit signals to the candle warming device to control activation and deactivation of the electric heating plate and the illumination symbols.

In another aspect, a remote-operated candle warmer device is disclosed. The device includes a housing accommodating an electric heating plate configured to warm a candle to diffuse fragrance without combustion, a power source including a rechargeable battery and an electrical cord with a plug for powering the electric heating plate, an illumination system comprising LED-based flame-like symbols disposed on the housing and operable independently of the electric heating plate, a control module configured to regulate the temperature of the electric heating plate and prevent overheating, and a signal receiver coupled to the control module and configured to receive wireless signals, wherein a handheld remote-control device is configured to transmit the wireless signals to the signal receiver to selectively control the activation of the electric heating plate, the illumination system, or both.

In another embodiment, a portable, remote-controlled candle warming device is disclosed. The device includes a housing with a cavity accommodating an electric heating plate for supporting and warming a candle. A power system including a rechargeable battery and an electrical cord with a plug for dual power options. A plurality of LED-based illumination symbols are disposed on the housing to visually signify operational status. A signal receiver is integrated into the housing and configured to receive wireless signals from a remote control. A remote-control device is configured to transmit wireless activation signals to the signal receiver. The housing and electric heating plate are dimensioned to accommodate candles of varying sizes and shapes.

In one embodiment, a method for remotely operating a candle warmer system is described. The method includes placing a candle on a warming device that includes an electric heating plate within a heat-resistant housing, powering the warming device using an internal rechargeable battery or an external electrical supply via an electric cord, activating the warming device by transmitting a wireless signal from a remote-control device to a signal receiver within the warming device, activating the electric heating plate in response to the wireless signal to warm the candle and release fragrance without combustion, illuminating flame-like symbols on the housing of the warming device to indicate its operational status, and deactivating the warming device by transmitting a subsequent wireless signal from the remote-control device.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of remote-operated candle warmer system of the present invention in accordance with the disclosed architecture;

FIG. 2 illustrates an exploded view of the candle warmer device of the present invention in accordance with the disclosed structure;

FIG. 3 illustrates a perspective view showing the candle warmer device being placed on a shelf and activated using the remote-control device in accordance with the disclosed structure;

FIG. 4 illustrates a perspective view of the candle warmer device being used for melting a candle in accordance with the disclosed structure;

FIG. 5 illustrates another embodiment of the remote-control device used in the remote-operated candle warmer system of the present invention in accordance with the disclosed structure; and

FIG. 6 illustrates a flow chart depicting a process of using the remote operated candle warmer system of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a candle warmer system that can be operated remotely to enhance safety and convenience for users. There is a long-felt need for a system that eliminates the risks associated with traditional open-flame candles while addressing the limitations of conventional candle warmers. Additionally, there is a long-felt need for a candle warming device that can be controlled remotely, reducing the potential for accidents caused by children or pets interacting with electrical cords or the device itself. Furthermore, there is a long-felt need for a system that provides safe use and energy efficiency. More specifically, there exists a long-felt need in the art for a remote-operated candle warmer system that offers wireless control, enabling users to safely and efficiently enjoy candle fragrances in a variety of environments. Finally, there is a long-felt need for a device that integrates modern technology, such as wireless remote operation and offers a much safer alternative to standard candle warmers.

The present invention, in one exemplary embodiment, is a remotely operated candle warmer system. The system comprises a portable candle warming device, the device includes a housing constructed with heat-resistant materials, an electric heating plate is disposed within the housing and configured to warm a candle placed thereon to release fragrance without combustion, an internal rechargeable battery provides electric power to the electric heating plate, an electric cord includes a plug for coupling the candle warming device to an external electrical supply, and a plurality of illumination symbols disposed on an exterior surface of the housing, the illumination symbols configured to indicate activation of the candle warming device, and a remote-control device is configured to wirelessly transmit signals to the candle warming device to control activation and deactivation of the electric heating plate and the illumination symbols.

Reference will now be made in detail to the present preferred embodiments of the invention, examples of which are illustrated in the accompanying drawings. Wherever possible, the same reference numerals are used in the drawings and the description to refer to the same or like parts.

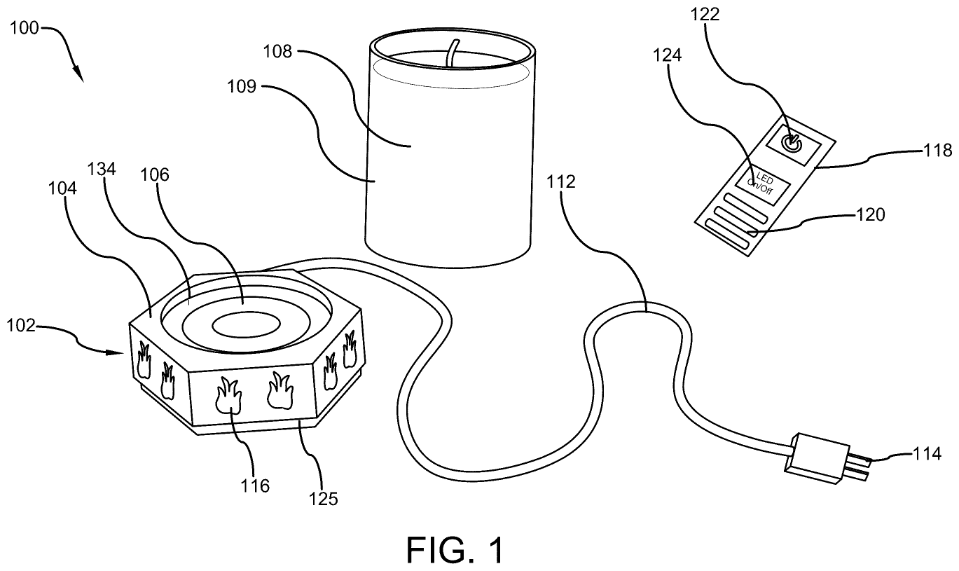

Referring initially to the drawings, FIG. 1 illustrates a perspective view of remote-operated candle warmer system of the present invention in accordance with the disclosed architecture. The remote-operated candle warmer system 100 of the present invention is designed to enable users to activate and control a candle warming device 102 remotely within a predetermined range. The system 100 uses remote control signals to activate a warming plate or electric heating plate to warm a wax candle 108 inside a glass jar surround 109 which releases fragrance into the air without burning the candle itself. The device 102 includes housing 104 for storing electronic components. The housing 102 also includes an electric heating plate 106 which is configured to gently warm the glass jar surround 109 and the wax candle 108 (i.e., the wax) contained therein to release fragrance without melting excessively or burning. The electric heating plate 106 maintains consistent and safe temperature levels. The housing 104 is constructed with heat-resistant materials such as high-grade ceramic or thermoplastic to provide safety and durability to users.

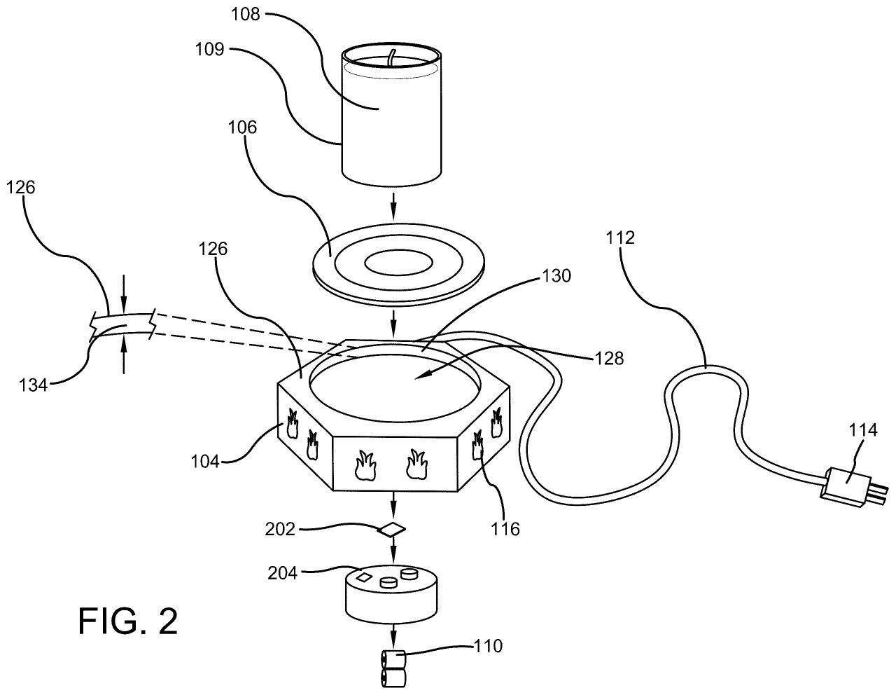

The housing 104 stores an internal rechargeable battery 110 (illustrated in FIG. 2) for providing electric power to the electric heating plate 106. The rechargeable battery 110 can be any Li-Ion battery and can be recharged while being placed inside the housing 104 or can also be removed from the housing 104. An electric cord 112 is also provided with the housing 104 which includes a 110-volt outlet plug 114 for coupling with conventional electrical supply.

A plurality of illumination symbols or indicia 116 are disposed in the exterior surface 118 of the housing 104. The illumination symbols or indicia 116 are made from LED lights or any other illumination sources for providing an indication of activation of the device 102 for warming the wax candle 108. Preferably, the illumination symbols or indicia 116 are in the form of flame-like visuals. When the electric power is supplied either by the battery 110 or through the electric cord 112, the heating plate 106 and the illumination symbols or indicia 116 may simultaneously activate and can also be individually controlled by a user.

For remotely operating the device 102, the system 100 includes a handheld remote-control device 118a which is configured to transmit wireless signals to the device 102 for controlling operation of the device 102. The remote-control device 118a preferably has a non-slip body 120 and a power button 122 is disposed therein. The power button 122 is adapted to activate and deactivate the candle warmer device 102 remotely when the remote-control device 118a and the candle warmer device 102 are within a predetermined range such as 10 meters. An independent illumination symbols control button 124 is configured to activate and deactivate the illumination symbols 116 independently of the heating plate 106. Upon activation of the device 102, the heating plate 106 is activated, and the glass jar surround 109 and wax candle 108 therein when placed on the heating plate 106, melts the wax 108 without open flames.

The candle warmer device 102 is portable, lightweight, and can be placed on distant shelves or hard-to-reach areas while controlled remotely using the remote-controlled device 118a. The system 100 can be used in various settings, such as homes, offices, or event spaces, to create a fragrant and pleasant atmosphere. Further, the bottom surface 125 of the housing 104 is non-slip and provides stability on different types of surfaces.

FIG. 2 illustrates an exploded view of the candle warmer device of the present invention in accordance with the disclosed structure. The device 102 includes an electric control module 202 coupled to the heating plate 106 for controlling and providing a uniform electrical supply to the heating plate 106 from the battery 110 and/or the electric cord 112. The electric control module 202 prevents overheating of the heating plate 106 and maintains uniform melting of the wax candle 108. A signal receiver 204 is configured to receive the wireless signal from the remote-control device 118a and process the signal into an instruction for activation and deactivation of heating plate 106 and/or the illumination symbols or indicia 116.

The housing 104 and the heating plate 106 can be of any shape and size to accommodate candles (i.e., glass jars) of different sizes. Further, the heating plate 106 can be made from aluminum or stainless steel with a non-stick coating for easy cleaning thereof.

Referring to FIGS. 1 and 2, the top surface 126 of the housing 104 is positioned above the heating plate 106 and more specifically, a cavity 128 of the housing 104 accommodates the heating plate 106. An electrically conducting slit 130 is disposed along the inner surface 132 of the housing 104 for securing the heating plate 106. A vertical gap 134 from about 0.5 inches to about 2 inches is disposed between the slit 130 and the top surface 126 of the housing 104, which also helps the candle from tilting and thereby provides a convenient operation of the smart candle warming device 100.

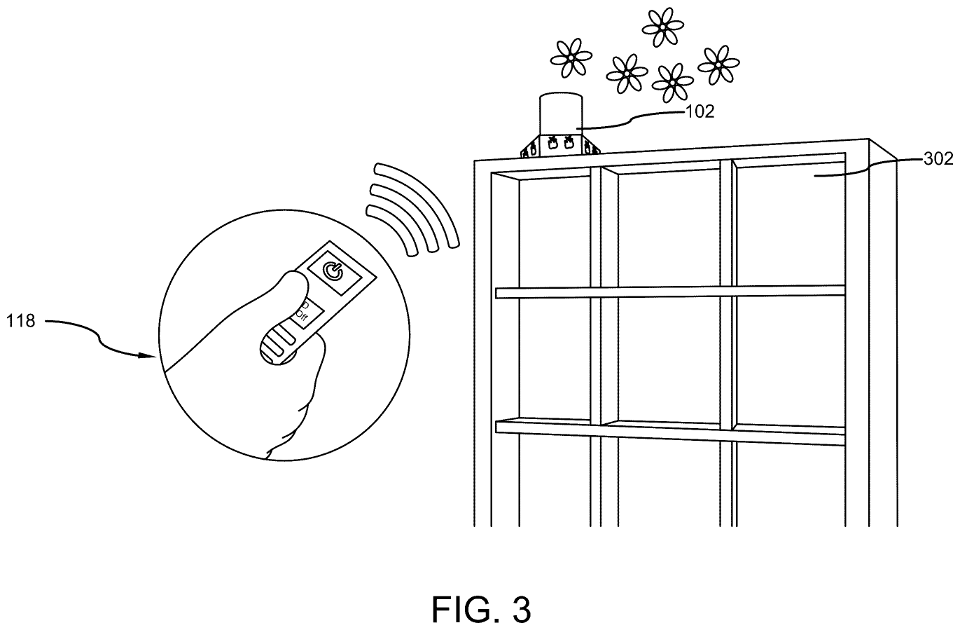

FIG. 3 illustrates a perspective view showing the candle warmer device being placed on a shelf and activated using the remote-control device in accordance with the disclosed structure. As illustrated, the candle warmer device 102 is placed on the shelf 302 and can be placed at any surface. In case, external power supply is not available, the internal battery 110 can be used for providing electric power to the heating plate 106 and the illumination symbols 116. The remote-control device 118a can be used by a user to remotely activate the heating plate 106 and the illumination symbols 116.

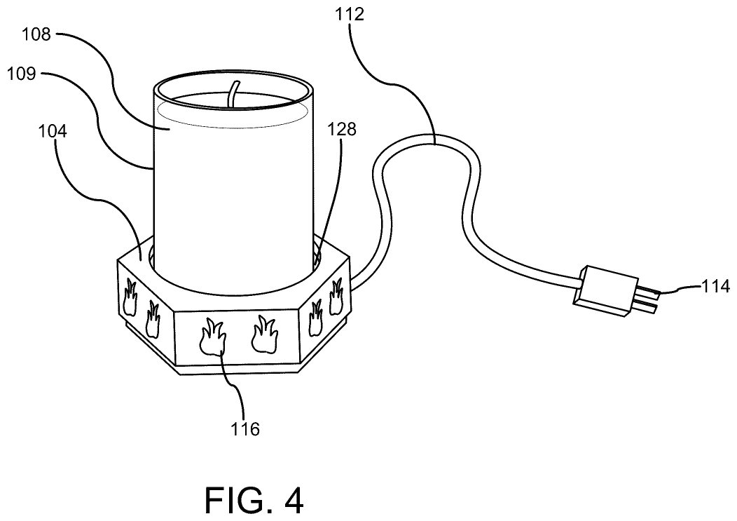

FIG. 4 illustrates a perspective view of the candle warmer device being used for melting a candle in accordance with the disclosed structure. In use, the glass jar surround 109 and the wax candle 108 therein, is securely placed on the heating plate 106 and is supported by the housing 102. In case, the plug 114 is inserted into conventional 110-Volt supply, the power is provided to the heating plate 106 and the illumination symbols 116 through the cord 112 and in other cases, the power is provided by the internal battery 110. In one embodiment, the heating plate 106 is heated to a temperature from 100 degrees to 190 degrees F. In another embodiment, the heating plate 106 is heated to a temperature from 120 degrees to 160 degrees F. In yet still another embodiment, the heating plate 106 is heated to a temperature from 130 degrees to 150 degrees F.

In some embodiments of the present invention, the heating plate 106 is temperature controlled including different heat settings based on different types of candles or wax products. The heating plate 106 temperature can be set or controlled based on the type of wax. For example, for soy wax the heat setting can be between 120 degrees and 140 degrees F. For paraffin wax, the heat setting can be between 170 degrees and 185 degrees F. For beeswax, the heat setting can be between 145 degrees and 155 degrees F. Further, the device 102 can be connected to smart home systems like Alexa, Google Assistant, or Apple Homekit for voice control and automation.

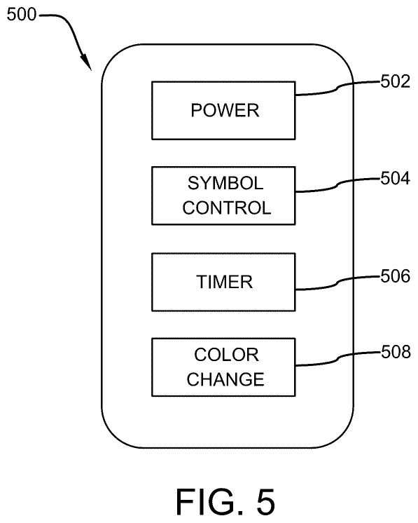

FIG. 5 illustrates another embodiment of the remote-control device used in the remote-operated candle warmer system of the present invention in accordance with the disclosed structure. In the present embodiment, the remote-control device 500 includes a power button 502, an illumination symbols control button 504, a timer button 506, and a color change button 508. The timer button 506 is configured to set a timing duration to automatically turn the warmer device 102 off after a specified duration. The color change button 508 enables users to customize the LED lighting within the flame symbols, enabling users to set different colors or patterns for ambiance.

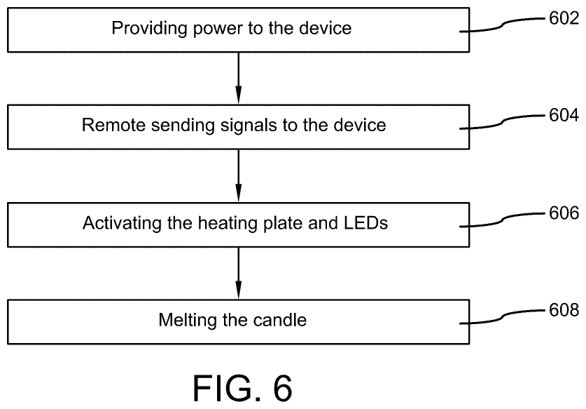

FIG. 6 illustrates a flow chart depicting a process of using the smart candle warming device of the present invention. Initially, a user places the glass jar surround 109 and the wax candle 108 on the heating plate 106 and the warmer device 102 is plugged into a standard 110-volt outlet or powered by the internal battery 110 (Step 602). Then, upon pressing the power button on the remote device, the remote sends a signal (RF/IR) to the warmer within the predefined range (Step 604). The warmer device 102 receives the signal and activates the heating plate and can activate the LED symbols to indicate the warmer device 102 is operational (Step 606). Thereafter, the heating plate 106 warms the glass jar surround 109 and the wax candle 108 therein, causing the fragrance of the wax candle 108 to diffuse into the air without burning (Step 608). In some embodiments, the warmer device 102 is automatically turned off after a specific duration and can also be manually turned off by the user.

In some embodiments, a software application installed in a smartphone, or any other similar electronic device can function as the remote-control device and provides the same functionality as provided by the remote-control device of different embodiments of the present invention.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “smart candle warming device”, “remote-operated candle warmer system”, “candle warmer system”, and “system” are interchangeable and refer to the remotely operated candle warmer system 100 of the present invention.

Notwithstanding the forgoing, the remotely operated candle warmer system 100 of the present invention can be of any suitable configuration as is known in the art without affecting the overall concept of the invention, provided that it accomplishes the above stated objectives. One of ordinary skill in the art will appreciate that the remotely operated candle warmer system 100 as shown in the FIGS. are for illustrative purposes only, and that many other configurations of the remotely operated candle warmer system 100 are well within the scope of the present disclosure. Although the dimensions of the remotely operated candle warmer system 100 are important design parameters for user convenience, the remotely operated candle warmer system 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A remote-operated candle warmer system comprising:
a handheld remote control;
a candle warming device having a housing, an electric heating plate; and
a wax candle having a glass jar surround;
wherein said handheld remote control remotely activates said electric heating plate;
wherein said electric heating plate maintains a consistent temperature level;
wherein said housing having a heat-resistant material selected from the group consisting of a high-grade ceramic and a thermoplastic; and
further wherein said heating plate is heated to a temperature from 100 degrees to 190 degrees F.

2. The remote-operated candle warmer system of claim 1, wherein said candle warming device having separate heat settings to control a temperature of said electric heating plate based on a type of wax in said wax candle.

3. The remote-operated candle warmer system of claim 2, wherein said type of wax is selected from the group consisting of a soy wax, a paraffin wax, and a beeswax.

4. The remote-operated candle warmer system of claim 3, said soy wax heat setting is between 120 degrees and 140 degrees F., said paraffin wax heat setting is between 170 degrees and 185 degrees F., and said beeswax heat setting is between 145 degrees and 155 degrees F.

5. The remote-operated candle warmer system of claim 1, wherein said heating plate is heated to a temperature from 120 degrees to 160 degrees F.

6. The remote-operated candle warmer system of claim 1, wherein said heating plate is heated to a temperature from 130 degrees to 150 degrees F.

7. The remote-operated candle warmer system of claim 5, wherein said housing having a rechargeable battery for providing electric power to said electric heating plate.

8. The remote-operated candle warmer system of claim 7, wherein said housing having an electric cord including a 110-volt outlet plug for coupling with an electrical supply.

9. The remote-operated candle warmer system of claim 8, wherein said handheld remote-control transmits wireless signals to said candle warming device for controlling operation of said candle warming device.

10. The remote-operated candle warmer system of claim 9, wherein said electric heating plate having a material selected from the group consisting of an aluminum and a stainless steel.

11. The remote-operated candle warmer system of claim 10, wherein said housing having a top surface, and further wherein said electric heating plate is positioned below said top surface.

12. The remote-operated candle warmer system of claim 11, wherein an inner surface of said housing having an electrically conducting slit disposed along a circumference of said inner surface of said housing for securing said electric heating plate.

13. The remote-operated candle warmer system of claim 12, wherein said electrically conducting slit and said top surface forming a vertical gap therebetween, and further wherein said vertical gap is from 0.5 inches to 2 inches.

14. A remote-operated candle warmer system comprising:
a handheld remote control;
a candle warming device having a housing, an electric heating plate; and
a wax candle having a glass jar surround;
wherein said handheld remote control remotely activates said electric heating plate;
wherein said electric heating plate maintains a consistent temperature level;
wherein said housing having a heat-resistant material selected from the group consisting of a high-grade ceramic and a thermoplastic; and
further wherein said candle warming device having separate heat settings to control a temperature of said electric heating plate based on a type of wax in said wax candle.

15. The remote-operated candle warmer system of claim 14, wherein said type of wax is selected from the group consisting of a soy wax, a paraffin wax, and a beeswax.

16. The remote-operated candle warmer system of claim 15, said soy wax heat setting is between 120 degrees and 140 degrees F., said paraffin wax heat setting is between 170 degrees and 185 degrees F., and said beeswax heat setting is between 145 degrees and 155 degrees F.

17. The remote-operated candle warmer system of claim 14, wherein said heating plate is heated to a temperature from 100 degrees to 190 degrees F.

18. A method of warming candle wax, the method comprising the steps of:
providing a candle warming device having a housing, an electric heating plate; and a wax candle including a glass jar surround, wherein said housing having a heat-resistant material selected from the group consisting of a high-grade ceramic and a thermoplastic;
activating said electric heating plate; and
maintaining a consistent temperature of said electric heating plate, wherein said heating plate is heated to a temperature from 100 degrees to 190 degrees F.

19. The method of warming candle wax of claim 18, wherein said candle warming device having separate heat settings to control a temperature of said electric heating plate based on a type of wax in said wax candle.

20. The method of warming candle wax of claim 19 further comprising a step of selecting said type of wax in said wax candle from the group consisting of a soy wax, a paraffin wax, and a beeswax, wherein said soy wax heat setting is between 120 degrees and 140 degrees F., said paraffin wax heat setting is between 170 degrees and 185 degrees F., and said beeswax heat setting is between 145 degrees and 155 degrees F.
